(19) United States
(12) Patent Application Publication
(10) Pub. No.: US 2013/0101690 A1
(43) Pub. Date: Apr. 25, 2013

(54) SUPPLEMENTS AND METHODS OF SUPPLEMENTATION

(71) Applicant: Shawn Talbott, Draper, UT (US)

(72) Inventor: Shawn Talbott, Draper, UT (US)

(73) Assignee: DBC, LLC, Lehi, UT (US)

(21) Appl. No.: 13/643,528

(22) PCT Filed: Apr. 27, 2011
(86) PCT No.: PCT/US11/34154

§ 371 Date: Dec. 27, 2012

Related U.S. Application Data
(60) Provisional application No. 61/328,980, filed on Apr. 28, 2010.

(51) Int. Cl. A23L 1/29; A61K 36/185; A61K 45/06; A61K 36/38; A61K 31/198; A61K 36/752; A61K 36/82

(52) U.S. Cl. CPC A23L 1/293; A61K 36/752; A61K 36/185; A61K 36/82; A61K 36/38; A61K 31/198; A61K 45/06
USPC 424/729; 426/648; 426/2

(57) ABSTRACT

The present invention provides supplements, combination supplements, methods of treating inflammation, methods of reducing tension, methods of increasing vigor, and methods of increasing metabolic balance. Further, the invention provides methods for supplementing the diet of an individual, and kits for use in supplementation. The supplements, combination supplements, methods and kits of the present invention may be used to achieve an outcome such as increased vigor, decreased tension, improved metabolic balance, and decreased levels of C-reactive protein.

### BACKGROUND

Chronic stress plays a major role in the pathophysiology of many disease states, particularly psychological disorders including depression, chronic fatigue syndrome, anxiety, fibromyalgia, and burnout. These stress-related changes in psychology may be due to both endocrine and behavioral factors, and may in some cases be mediated or attenuated by lifestyle factors including stress management, diet, exercise, and dietary supplements. Some characterize the state of “Vigor” as presenting itself as a 3-tiered sustained mood-state that is characterized by (1) physical energy, (2) mental acuity, and (3) cognitive liveliness. While vigor exists on a continuum of mood states, its antithesis is “burnout,” characterized by some as a state of physical fatigue, mental exhaustion, and cognitive weariness.

In both men and women, a balance between cortisol and testosterone is needed to build muscle and immune system components, as well as to control many aspects of physiology, including blood cell production and metabolism of protein, carbohydrates, and fat from food. A rise in cortisol exposure and/or a drop in testosterone in both men and women may lead to fatigue, a loss of sex drive, and abdominal adipose accumulation.

It is well described in the scientific and medical literature that male or female subjects who have low levels of testosterone are more likely to be depressed than those with normal testosterone levels. When testosterone levels are brought back to normal levels, mood also returns back to normal levels. Dozens of studies show that maintaining testosterone levels at more “youthful” levels (that is, keeping them from dropping with age) is associated with numerous health benefits in both men and women. For example, men and women with low testosterone may develop abdominal obesity (belly fat), a loss in sex drive, and become depressed.

In addition to the above, inflammation is emerging as a predictor of cardiovascular disease and may be considered a precursor to metabolic syndrome. Therefore the ability to reduce inflammation may be valuable in preventing the progression to diabetes and heart disease.

There currently exist several pharmaceutical options for treatment of inflammation, including NSAIDS. There are, however, potentially harmful side effects associated with their use. Dietary supplements represent a potential treatment option that may offer anti-inflammatory benefit with a lower risk profile.

To this end, there is a need for dietary supplements and methods of administering dietary supplements to promote wellness, and more specifically, to serve as non-pharmaceutical alternatives for the improvement of mood and metabolic balance, and for the treatment of inflammation.

### BRIEF DESCRIPTION OF THE INVENTION

The purpose of this invention is to provide non-pharmaceutical alternatives to improve mood and metabolic balance, and to decrease inflammation. The invention provides supplements for the treatment of these conditions, methods of treating inflammation, methods of reducing tension, methods of increasing vigor, and methods of increasing metabolic balance. Further, the invention provides methods for supplementing the diet of an individual, and kits for use in such supplementation.

In some embodiments, the invention provides a combination supplement comprising Citrus sinensis extract, Eurycoma longifolia extract, Camellia sinensis extract, L-theanine, pericarp from the fruit of a Garcinia mangostana L. tree, and juice from the fruit of a Garcinia mangostana L. tree. In one such embodiment, the combination supplement comprises a plurality of dietary supplements.

In another embodiment, the combination supplement comprises a first dietary supplement and a second dietary supplement.

In one such embodiment, the first dietary supplement comprises Citrus sinensis extract, Eurycoma longifolia extract, Camellia sinensis extract, and L-theanine, and wherein the second dietary supplement comprises pericarp from the fruit of a Garcinia mangostana L. tree, and juice from the fruit of a Garcinia mangostana L. tree.

In another such embodiment, the first dietary supplement comprises about 23% to about 30% by weight Citrus sinensis extract. In another such embodiment, the first dietary supplement comprises about 6% to about 15% by weight Eurycoma longifolia extract. In another such embodiment, the first dietary supplement comprises about 15% to about 23% by weight Camellia sinensis extract. In another such embodiment, the first dietary supplement comprises about 6% to about 15% by weight L-theanine. In another such embodiment, the second dietary supplement comprises about 3% to about 50% by weight pericarp from the fruit of a Garcinia mangostana L. tree. In another such embodiment, the second dietary supplement further comprises a fruit juice or a vegetable juice.

In a preferred embodiment, the combination supplement comprises Eleviv™, and XanGo® juice.

Another embodiment of the present invention provides methods for treating an individual to achieve an outcome selected from the group consisting of increased vigor, decreased tension, improved metabolic balance, and decreased C-reactive protein (CRP), comprising administering to the individual an effective amount of a combination supplement comprising Citrus sinensis extract, Eurycoma longifolia extract, Camellia sinensis extract, L-theanine, pericarp from the fruit of a Garcinia mangostana L. tree, and juice from the fruit of a Garcinia mangostana L. tree.

In one such embodiment, the combination supplement comprises a plurality of dietary supplements.

In another such embodiment, the combination supplement comprises a first dietary supplement and a second dietary supplement.

In one such embodiment, the first dietary supplement comprises Citrus sinensis extract, Eurycoma longifolia extract, Camellia sinensis extract, and L-theanine, and wherein the second dietary supplement comprises pericarp from the fruit of a Garcinia mangostana L. tree, and juice from the fruit of a Garcinia mangostana L. tree.

In one such embodiment, the first dietary supplement comprises about 23% to about 30% by weight Citrus sinensis extract. In another such embodiment, the first dietary supplement comprises about 6% to about 15% by weight Eurycoma longifolia extract. In another such embodiment, the first dietary supplement comprises about 15% to about 23% by weight Camellia sinensis extract. In another such embodiment, the first dietary supplement comprises about 6% to about 15% by weight L-theanine. In another such embodiment, the second dietary supplement comprises about 3% to about 50% by weight pericarp from the fruit of a Garcinia mangostana L. tree. In another such embodiment, the second dietary supplement further comprises a fruit juice or a vegetable juice.

In a preferred embodiment the method comprises administering Eleviv and XanGo juice.

In one such embodiment, Eleviv is administered once a day, and XanGo juice is administered twice a day.

In another such embodiment, three ounces of XanGo juice and two capsules of Eleviv are administered before noon on the day of administration, three ounces of XanGo juice are administered after noon on the day of administration.

Another embodiment of the present invention provides a method of supplementing the diet of an individual comprising administering to the individual a combination supplement comprising Citrus sinensis extract, Eurycoma longifolia extract, Camellia sinensis extract, L-theanine, pericarp from the fruit of a Garcinia mangostana L. tree, and juice from the fruit of a Garcinia mangostana L. tree.

In one such embodiment, the combination supplement comprises a plurality of dietary supplements.

In another such embodiment, the combination supplement comprises a first dietary supplement and a second dietary supplement.

In one such embodiment, the first dietary supplement comprises Citrus sinensis extract, Eurycoma longifolia extract, Camellia sinensis extract, and L-theanine, and wherein the second dietary supplement comprises pericarp from the fruit of a Garcinia mangostana L. tree, and juice from the fruit of a Garcinia mangostana L. tree.

In one such embodiment, the first dietary supplement comprises about 23% to about 30% by weight Citrus sinensis extract. In another such embodiment, the first dietary supplement comprises about 6% to about 15% by weight Eurycoma longifolia extract. In another such embodiment, the first dietary supplement comprises about 15% to about 23% by weight Camellia sinensis extract. In another such embodiment, the first dietary supplement comprises about 6% to about 15% by weight L-theanine. In another such embodiment, the second dietary supplement comprises about 3% to about 50% by weight pericarp from the fruit of a Garcinia mangostana L. tree. In another such embodiment, the second dietary supplement further comprises a fruit juice or a vegetable juice.

In a preferred embodiment the method comprises administering Eleviv and XanGo juice.

In one such embodiment, Eleviv is administered once a day, and XanGo juice is administered twice a day.

In another such embodiment, three ounces of XanGo juice and two capsules of Eleviv are administered before noon on the day of administration, three ounces of XanGo Juice are administered after noon on the day of administration.

Another embodiment of the present invention provides a kit comprising a combination supplement and instructions for use of the combination supplement, wherein the combination supplement comprises Citrus sinensis extract, Eurycoma longifolia extract, Camellia sinensis extract, L-theanine, pericarp from the fruit of a Garcinia mangostana L. tree, and juice from the fruit of a Garcinia mangostana L. tree.

In one such embodiment, the combination supplement comprises a plurality of dietary supplements.

In another such embodiment, the kit comprises a first dietary supplement and a second dietary supplement.

In one such embodiment, the first dietary supplement comprises Citrus sinensis extract, Eurycoma longifolia extract, Camellia sinensis extract, and L-theanine, and wherein the second dietary supplement comprises pericarp from the fruit of a Garcinia mangostana L. tree, and juice from the fruit of a Garcinia mangostana L. tree.

In one such embodiment, the first dietary supplement comprises about 23% to about 30% by weight Citrus sinensis extract. In another such embodiment, the first dietary supplement comprises about 6% to about 15% by weight Eurycoma longifolia extract. In one such embodiment, the first dietary supplement comprises about 15% to about 23% by weight Camellia sinensis extract. In one such embodiment, the first dietary supplement comprises about 6% to about 15% by weight L-theanine. In one such embodiment, the second dietary supplement comprises about 3% to about 50% by weight pericarp from the fruit of a Garcinia mangostana L. tree. In one such embodiment, the second dietary supplement further comprises a fruit juice or a vegetable juice. In one such embodiment, the first dietary supplement is Eleviv, and wherein the second dietary supplement is XanGo juice.

### BRIEF DESCRIPTION OF THE DRAWINGS

FIG. 1 depicts the average change in cortisol and the change in cortisol: testosterone (C:T) ratio in individual treated with Eleviv for 8 or 12 weeks

FIG. 2 depicts the average change in tension, fatigue, confusion, depression, anger and vigor as measured by the Profile of Mood States in individuals treated with Eleviv for 8 or 12 weeks.

### DETAILED DESCRIPTION OF PREFERRED EMBODIMENTS

The present invention provides combination supplements, methods of treating inflammation, methods of reducing tension, methods of increasing vigor, and methods of increasing metabolic balance. Further, the invention provides methods for supplementing the diet of an individual, and kits for use in supplementation. The combination supplements, methods and kits of the present invention may be used to achieve an outcome such as increased vigor, decreased tension, improved metabolic balance, and decreased CRP.

### Definitions

“Natural products” are defined as any composition found in nature that can be ingested by an individual to cause an effect in that individual. Natural products may be whole items (e.g. a whole fruit), or they may be an isolated component of an item (e.g. a hot water extract of a plant). Natural products may be processed to aid in consumption (e.g. ground up).

“Dietary Supplement” is defined as a non-pharmaceutical item for consumption that comprises one or more natural products formulated together.

“Combination supplement” is defined as one or more dietary supplements which are administered to an individual. The individual dietary supplements which make up the combination supplement are administered in such a way that at least one of the natural products of the combination supplement has a greater effect than if the natural product were ingested alone. The individual dietary supplements of a combination supplement may be formulated separately.

“Metabolic Balance” is defined as the balance between anabolic to catabolic hormones in an individual. More specifically, metabolic balance refers to the balance between testosterone and cortisol in an individual. Metabolic balance may be expressed as a ratio of cortisol to testosterone or Free Androgen Index, or vice versa. An increase in metabolic balance means that either the level of testosterone in an individual increases relative to the level of cortisol, or the level of cortisol decreases relative to the level of testosterone, or both. Thus, when metabolic balance is expressed as a ratio of cortisol to testosterone or cortisol to Free Androgen index, an increase in metabolic balance is evidenced by a decrease in the ratio (i.e. negative percent change). Hormone levels may be assayed by testing salivary or blood testosterone levels, the Free Androgen index, or (cortisol levels measured in saliva, urine or blood).

The terms “Tension,” “Fatigue,” “Confusion,” “Depression,” “Anger,” and “Vigor” have the meaning as defined in the Profile of Mood States (POMS), developed by Maurice Lorr, Ph.D., Douglas M. McNair, Ph.D. and Leo F. Droppleman, Ph.D. The definition of these terms and the methods of measuring them are described in detail in the POMS documentation, including the POMS standard kit, which is incorporated by reference herein in its entirety. The POMS standard kit (POM28) is available for purchase from Multi-Health Systems, Inc. (www.mhs.com).

“Inflammation,” which is well defined in the prior art, is at least in some instances defined as an immune reaction characterized by activation of white blood cells and the release of cytokines. C-reactive protein (CRP) is often used as an indicator of inflammation, particularly with respect to the inflammatory aspects of cardiovascular disease. CRP levels can be measured using methods known to one skilled in the art, such as the HS-CRP test that is well known in the art.

### Combination Supplements

The combination supplements of the present invention may be used to promote wellness by improving mood, improving metabolic balance, and by reducing inflammation. More specifically, the combination supplements may be used to increase vigor, reduce tension, increase the ratio of testosterone to cortisol, and decrease the level of CRP in an individual.

In one embodiment, the combination supplements comprise Citrus sinensis extract, Eurycoma longifolia extract, Camellia sinensis extract, L-theanine, pericarp from the fruit of a Garcinia mangostana L. tree, and juice from the fruit of a Garcinia mangostana L. tree. In some embodiments, the combination supplement is a single dietary supplement which comprises: Citrus sinensis extract, Eurycoma longifolia extract, Camellia sinensis extract, L-theanine, pericarp from the fruit of a Garcinia mangostana L. tree, and juice from the fruit of a Garcinia mangostana L. tree. In other embodiments, the combination supplement is comprised of a plurality of dietary supplements each comprising at least one of: Citrus sinensis extract, Eurycoma longifolia extract, Camellia sinensis extract, L-theanine, pericarp from the fruit of a Garcinia mangostana L. tree, and juice from the fruit of a Garcinia mangostana L. tree, such that the ingredients of the plurality of dietary supplements together comprises at least Citrus sinensis extract, Eurycoma longifolia extract, Camellia sinensis extract, L-theanine, pericarp from the fruit of a Garcinia mangostana L. tree, and juice from the fruit of a Garcinia mangostana L. tree.

In some embodiments, the combination supplement is comprised of two dietary supplements. In one such embodiment, there are two dietary supplements, the first dietary supplement comprising Citrus sinensis extract, Eurycoma longifolia extract, Camellia sinensis extract, and L-theanine; and the second dietary supplement comprising pericarp from the fruit of a Garcinia mangostana L. tree, and juice from the fruit of a Garcinia mangostana L. tree. In a further embodiment, there are two dietary supplements, the first dietary supplement comprising: Citrus sinensis extract, Eurycoma longifolia extract, Camellia sinensis extract, and L-theanine, and the second dietary supplement comprising: pericarp from the fruit of a Garcinia mangostana L. tree, juice from the fruit of a Garcinia mangostana L. tree, and an additional fruit or vegetable juice.

In another such embodiment, the second dietary supplement comprises about 3% to about 50% by weight pericarp from the fruit of a Garcinia mangostana L. tree. In another such embodiment, the second dietary supplement comprises about 5% to about 25% by weight pericarp from the fruit of a Garcinia mangostana L. tree. In another such embodiment, the second dietary supplement comprises about 10% to about 20% by weight pericarp from the fruit of a Garcinia mangostana L. tree.

In another such embodiment, the first dietary supplement comprises about 23% to about 30% by weight Citrus sinensis extract. In another such embodiment a dietary supplement comprises about 6% to about 15% by weight Eurycoma longifolia extract. In another such embodiment, the first dietary supplement comprises about 15% to about 23% by weight Camellia sinensis extract. In another such embodiment the first dietary supplement comprises about 6% to about 15% by weight L-theanine.

In one embodiment, a dietary supplement may be provided in a liquid form. In another embodiment, a dietary supplement may be provided as a juice. In other embodiments, a dietary supplement may be provided as a capsule, pill, or other orally ingestible form. In a preferred embodiment, the dietary supplement comprises Eleviv™ and XanGo® juice. Eleviv is a proprietary blend of Citrus sinensis extract, Eurycoma longifolia extract, Camellia sinensis extract, and L-theanine sold by XanGo, LLC. XanGo juice is a proprietary blend of pericarp from the fruit of a Garcinia mangostana L. tree, juice from the fruit of a Garcinia mangostana L. tree, and additional juices sold by XanGo LLC.

In another embodiment, the dietary supplement or dietary supplements may be provided as a kit comprising one or more dietary supplements and instructions for their use.

Citrus sinensis extract is prepared by extracting the peel of Citrus Sinensis with hot water. Citrus sinensis extract includes Polymethoxylated Flavones (PMFs), particularly tangeretin, sinensetin, and nobilitin, which are known to have health benefits in reducing cholesterol and promoting cardiovascular health. In some embodiments Citrus sinensis extract is prepared such that the extract comprises about 30% by weight PMFs.

Eurycoma longifolia extract is prepared by extracting the root of Eurycoma longifolia with hot water. Eurycoma longifolia extract includes Glycosaponins and Eurypeptides. In some embodiments Eurycoma longifolia extract is prepared such that the extract comprises about 40% by weight Glycosaponins and about 22% Eurypeptides. Eurypeptides are defined herein as peptides derived from Eurycoma longifolia. These peptides may be extracted, for example, by boiling pulverized Eurycoma longifolia root in water. Additional disclosure regarding methods of extracting and the identity of Eurypeptides can be found in U.S. Pat. No. 7,132,117, which is hereby incorporated by reference in its entirety.

Camellia sinensis extract is prepared by extracting the leaves of Camellia sinensis with hot water. In some embodiments Camellia sinensis extract includes polyphenols, catechins, and epigallocatechin gallate (EGCG). Camellia sinensis extract is prepared such that the extract comprises about 50% Polyphenols, about 30% Catechins, and about 15% EGCG

Pericarp from the fruit of a Garcinia mangostana L. tree may be prepared by removing the thick pericarp of the fruit from the inner pulp. Juice may additionally be extracted from the pulp. In one embodiment, the pericarp and the juice from the fruit of a Garcinia mangostana L. tree are prepared together. In this embodiment, whole fruit from a Garcinia mangostana L. tree is picked and then ground into a pulp and pericarp mixture using commercially available grinding equipment. Selected juice concentrates and water may optionally be added. Methods of preparing juice and pericarp from a Garcinia mangostana L. tree are additionally disclosed in U.S. Pat. No. 6,730, 333, which is hereby incorporated by reference in its entirety.

### Methods of Treating to Achieve Increased Vigor

Another embodiment of the present invention provides methods for treating an individual to achieve an outcome of increased vigor. One embodiment of this method comprises administering to the individual an effective amount of a combination supplement comprising Citrus sinensis extract, Eurycoma longifolia extract, Camellia sinensis extract, L-theanine, pericarp from the fruit of a Garcinia mangostana L. tree, and juice from the fruit of a Garcinia mangostana L. tree.

In one such embodiment, the combination supplement comprises a plurality of dietary supplements.

In another such embodiment, the combination supplement comprises a first dietary supplement and a second dietary supplement.

In one such embodiment, the first dietary supplement comprises Citrus sinensis extract, Eurycoma longifolia extract, Camellia sinensis extract, and L-theanine, and wherein the second dietary supplement comprises pericarp from the fruit of a Garcinia mangostana L. tree, and juice from the fruit of a Garcinia mangostana L. tree.

In one such embodiment, the first dietary supplement comprises about 23% to about 30% by weight Citrus sinensis extract. In another such embodiment, the first dietary supplement comprises about 6% to about 15% by weight Eurycoma longifolia extract. In another such embodiment, the first dietary supplement comprises about 15% to about 23% by weight Camellia sinensis extract. In another such embodiment, the first dietary supplement comprises about 6% to about 15% by weight L-theanine. In another such embodiment, the second dietary supplement comprises about 3% to about 50% by weight pericarp from the fruit of a Garcinia mangostana L. tree. In another such embodiment, the second dietary supplement further comprises at least one additional fruit or vegetable juice.

In a preferred embodiment the method comprises administering Eleviv and XanGo juice.

In one such embodiment, Eleviv is administered once a day, and XanGo juice is administered twice a day.

In another such embodiment, three ounces of XanGo juice and two capsules of Eleviv are administered before noon on the day of administration, three ounces of XanGo juice are administered after noon on the day of administration.

### Methods of Treating to Achieve Increased Metabolic Balance

Another embodiment of the present invention provides methods for treating an individual to achieve an outcome of increased metabolic balance. One embodiment of this method comprises administering to the individual an effective amount of a combination supplement comprising Citrus sinensis extract, Eurycoma longifolia extract, Camellia sinensis extract, L-theanine, pericarp from the fruit of a Garcinia mangostana L. tree, and juice from the fruit of a Garcinia mangostana L. tree.

In one such embodiment, the combination supplement comprises a plurality of dietary supplements.

In another such embodiment, the combination supplement comprises a first dietary supplement and a second dietary supplement.

In one such embodiment, the first dietary supplement comprises Citrus sinensis extract, Eurycoma longifolia extract, Camellia sinensis extract, and L-theanine, and wherein the second dietary supplement comprises pericarp from the fruit of a Garcinia mangostana L. tree, and juice from the fruit of a Garcinia mangostana L. tree.

In one such embodiment, the first dietary supplement comprises about 23% to about 30% by weight Citrus sinensis extract. In another such embodiment, the first dietary supplement comprises about 6% to about 15% by weight Eurycoma longifolia extract. In another such embodiment, the first dietary supplement comprises about 15% to about 23% by weight Camellia sinensis extract. In another such embodiment, the first dietary supplement comprises about 6% to about 15% by weight L-theanine. In another such embodiment, the second dietary supplement comprises about 3% to about 50% by weight pericarp from the fruit of a Garcinia mangostana L. tree. In another such embodiment, the second dietary supplement further comprises a fruit juice or a vegetable juice.

In a preferred embodiment the method comprises administering Eleviv and XanGo juice.

In one such embodiment, Eleviv is administered once a day, and XanGo juice is administered twice a day.

In another such embodiment, three ounces of XanGo Juice and two capsules of Eleviv are administered before noon on the day of administration, three ounces of XanGo juice are administered after noon on the day of administration.

### Methods of Treating to Achieve Decreased Tension

Another embodiment of the present invention provides methods for treating an individual to achieve an outcome of decreased tension. One embodiment of this method comprises administering to the individual an effective amount of a combination supplement comprising Citrus sinensis extract, Eurycoma longifolia extract, Camellia sinensis extract, L-theanine, pericarp from the fruit of a Garcinia mangostana L. tree, and juice from the fruit of a Garcinia mangostana L. tree.

In one such embodiment, the combination supplement comprises a plurality of dietary supplements.

In another such embodiment, the combination supplement comprises a first dietary supplement and a second dietary supplement.

In one such embodiment, the first dietary supplement comprises Citrus sinensis extract, Eurycoma longifolia extract, Camellia sinensis extract, and L-theanine, and wherein the second dietary supplement comprises pericarp from the fruit of a Garcinia mangostana L. tree, and juice from the fruit of a Garcinia mangostana L. tree.

In one such embodiment, the first dietary supplement comprises about 23% to about 30% by weight Citrus sinensis extract. In another such embodiment, the first dietary supplement comprises about 6% to about 15% by weight Eurycoma longifolia extract. In another such embodiment, the first dietary supplement comprises about 15% to about 23% by weight Camellia sinensis extract. In another such embodiment, the first dietary supplement comprises about 6% to about 15% by weight L-theanine. In another such embodiment, the second dietary supplement comprises about 3% to about 50% by weight pericarp from the fruit of a Garcinia mangostana L. tree. In another such embodiment, the second dietary supplement further comprises a fruit juice or a vegetable juice.

In a preferred embodiment the method comprises administering Eleviv and XanGo juice.

In one such embodiment, Eleviv is administered once a day, and XanGo juice is administered twice a day.

In another such embodiment, three ounces of XanGo juice and two capsules of Eleviv are administered before noon on the day of administration, three ounces of XanGo juice are administered after noon on the day of administration.

### Methods of Treating to Achieve Decreased Inflammation

Another embodiment of the present invention provides methods for treating an individual to achieve an outcome of decreased inflammation. One embodiment of this method comprises administering to the individual an effective amount of a combination supplement comprising Citrus sinensis extract, Eurycoma longifolia extract, Camellia sinensis extract, L-theanine, pericarp from the fruit of a Garcinia mangostana L. tree, and juice from the fruit of a Garcinia mangostana L. tree.

In one such embodiment, the combination supplement comprises a plurality of dietary supplements.

In another such embodiment, the combination supplement comprises a first dietary supplement and a second dietary supplement.

In one such embodiment, the first dietary supplement comprises Citrus sinensis extract, Eurycoma longifolia extract, Camellia sinensis extract, and L-theanine, and wherein the second dietary supplement comprises pericarp from the fruit of a Garcinia mangostana L. tree, and juice from the fruit of a Garcinia mangostana L. tree.

In one such embodiment, the first dietary supplement comprises about 23% to about 30% by weight Citrus sinensis extract. In another such embodiment, the first dietary supplement comprises about 6% to about 15% by weight Eurycoma longifolia extract. In another such embodiment, the first dietary supplement comprises about 15% to about 23% by weight Camellia sinensis extract. In another such embodiment, the first dietary supplement comprises about 6% to about 15% by weight L-theanine. In another such embodiment, the second dietary supplement comprises about 3% to about 50% by weight pericarp from the fruit of a Garcinia mangostana L. tree. In another such embodiment, the second dietary supplement further comprises a fruit juice or a vegetable juice.

In a preferred embodiment the method comprises administering Eleviv and XanGo juice.

In one such embodiment, Eleviv is administered once a day, and XanGo juice is administered twice a day.

In another such embodiment, three ounces of XanGo Juice and two capsules of Eleviv are administered before noon on the day of administration, three ounces of XanGo juice are administered after noon on the day of administration.

### Methods of Supplementing the Diet of an Individual

Another embodiment of the present invention provides a method of supplementing the diet of an individual comprising administering to the individual a combination supplement comprising Citrus sinensis extract, Eurycoma longifolia extract, Camellia sinensis extract, L-theanine, pericarp from the fruit of a Garcinia mangostana L. tree, and juice from the fruit of a Garcinia mangostana L. tree.

In one such embodiment, the combination supplement comprises a plurality of dietary supplements.

In another such embodiment, the combination supplement comprises a first dietary supplement and a second dietary supplement.

In one such embodiment, the first dietary supplement comprises Citrus sinensis extract, Eurycoma longifolia extract, Camellia sinensis extract, and L-theanine, and wherein the second dietary supplement comprises pericarp from the fruit of a Garcinia mangostana L. tree, and juice from the fruit of a Garcinia mangostana L. tree.

In one such embodiment, the first dietary supplement comprises about 23% to about 30% by weight Citrus sinensis extract. In another such embodiment, the first dietary supplement comprises about 6% to about 15% by weight Eurycoma longifolia extract. In another such embodiment, the first dietary supplement comprises about 15% to about 23% by weight Camellia sinensis extract. In another such embodiment, the first dietary supplement comprises about 6% to about 15% by weight L-theanine. In another such embodiment, the second dietary supplement comprises about 3% to about 50% by weight pericarp from the fruit of a Garcinia mangostana L. tree. In another such embodiment, the second dietary supplement further comprises a fruit juice or a vegetable juice.

In a preferred embodiment the method comprises administering Eleviv and XanGo juice.

In one such embodiment, Eleviv is administered once a day, and XanGo juice is administered twice a day.

In another such embodiment, three ounces of XanGo juice and two capsules of Eleviv are administered before noon on the day of administration, three ounces of XanGo juice are administered after noon on the day of administration.

### Kits for Use in Supplementing the Diet

Another embodiment of the present invention provides a kit comprising a combination supplement and instructions for use of the combination supplement, wherein the combination supplement comprises Citrus sinensis extract, Eurycoma longifolia extract, Camellia sinensis extract, L-theanine, pericarp from the fruit of a Garcinia mangostana L. tree, and juice from the fruit of a Garcinia mangostana L. tree.

In one such embodiment, the combination supplement comprises a plurality of dietary supplements.

In another such embodiment, the kit comprises a first dietary supplement and a second dietary supplement.

In one such embodiment, the first dietary supplement comprises Citrus sinensis extract, Eurycoma longifolia extract, Camellia sinensis extract, and L-theanine, and wherein the second dietary supplement comprises pericarp from the fruit of a Garcinia mangostana L. tree, and juice from the fruit of a Garcinia mangostana L. tree.

In one such embodiment, the first dietary supplement comprises about 23% to about 30% by weight Citrus sinensis extract. In another such embodiment, the first dietary supplement comprises about 6% to about 15% by weight Eurycoma longifolia extract. In one such embodiment, the first dietary supplement comprises about 15% to about 23% by weight Camellia sinensis extract. In one such embodiment, the first dietary supplement comprises about 6% to about 15% by weight L-theanine. In one such embodiment, the second dietary supplement comprises about 3% to about 50% by weight pericarp from the fruit of a Garcinia mangostana L. tree. In one such embodiment, the second dietary supplement further comprises at least one fruit or vegetable juice. In one such embodiment, the first dietary supplement is Eleviv, and wherein the second dietary supplement is XanGo juice.

### Example 1

### Supplementation with Eleviv

### Procedures

We recruited 82 self-reported “high-stress” volunteers. Fifty participated in an 8-week lifestyle intervention program and 32 participated in a 12-week intervention. Each program included education about stress management, nutrition intervention, exercise recommendations, and dietary supplementation (a non-commercial combination of Eurycoma/Citrus sinensis/Camellia sinensis). The entire lifestyle intervention program, called “SENSE” to denote each aspect of the program—Stress management, Exercise, Nutrition, Supplementation, and Evaluation—is available at www.CortisolConnection.com.

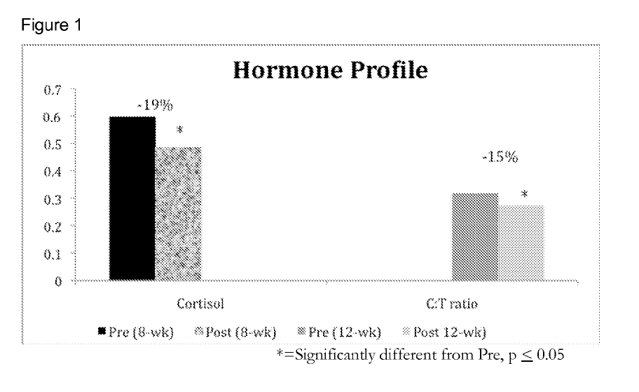

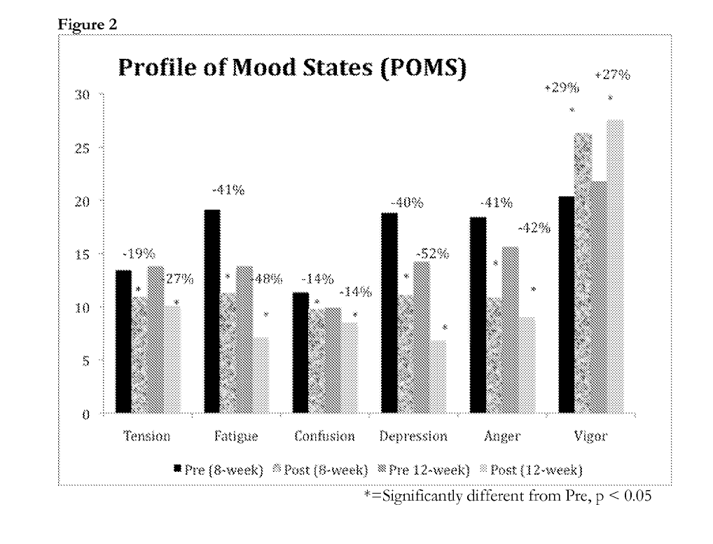

Before & after each lifestyle intervention program, we measured body weight, body fat, salivary cortisol and testosterone (C and T), & Psychological Mood State (assessed by Profile of Mood States). Baseline characteristics of each subject cohort (8-wk and 12-wk intervention) are shown in Table 1. Results showed that the lifestyle intervention program, in both 8-week and 12-week iterations, led to significant changes in body weight and body fat (data not shown), C:T ratio (FIG. 1), and Psychological Mood State scores (FIG. 2, all p<0.05 compared to baseline values).

TABLE 1
Baseline Characteristics

| | Age (y) | BW (kg) | BF (%) | BMI |
|---|---|---|---|---|
| 8-week Intervention | 44 ± 7 | 78.5 ± 16.3 | 32.4% ± 6.9 | 28.8 ± 4.8 |
| N = 46 compete (of 50) | | | | |
| 12-week Intervention | 44 ± 8 | 84.1 ± 39.5 | 35.1 ± 9.9 | 29.5 ± 5.6 |
| N = 29 complete (of 32) | | | | |

These findings show that even individuals self-described as “high-stress” can derive significant benefits in maintaining metabolic hormone balance and improving psychological mood state (both of which tend to become disrupted by chronic stress). Our data demonstrate that restoring metabolic hormone balance (e.g. −15% C:T ratio) from a state of “imbalance” (caused by chronic stress overexposure) is effective in improving measures of vigor (+27/+29%), depression (−40/−52%), fatigue (−41/−48%), and confusion (−14%/−14%).

### Example 2

### Supplementation with XanGo Juice

### Study Design

The study was an 8 week randomized, double-blind, placebo-controlled study with a pre-study 2 week washout period. The study was conducted at a single site: Medicus Research, LLC, in Northridge, Calif. Sample size was not calculated as this was an exploratory trial to compare three concentrations of the target product to placebo. The study was approved by the Copernicus Group IRB (Cary, N.C.) prior to the initiation of the study.

### Recruitment

Subjects were serially recruited if they were between 30-75 years of age, had a body mass index (BMI) ≧30 and ≦45 kg/m2 (obese), a HS-CRP of ≧3, agreed to discontinue anti-inflammatory medications and supplements (other than daily 81 mg aspirin which was allowed), agreed to use approved birth control methods if a female of childbearing age, and agreed to not initiate or change any exercise or diet programs during the study.

Subjects were excluded if they had consumed the test product in the past, had allergies to the test product, using any drugs that can affect CRP, were taking hormone replacements, anticoagulant or anti-platelet therapy, had surgery in the past 6 months, smoked cigarettes, known alcohol or drug abuse, had major systemic, inflammatory or chronic disease, untreated depression, active eating disorder, were unable to understand or follow study protocol, were pregnant or lactating and had any medical condition which in the opinion of the investigator might interfere with the subject's ability in the trial.

### Clinical Study Process

The subjects in the study came to the research clinic for a total of 4 visits (V0-V3). At V0, the screening visit, subjects were screened for eligibility according to the inclusion/exclusion criteria. Subjects gave their informed written consent before any procedures were conducted.

There was a 2 week washout phase between screening and enrollment during which subjects were asked to refrain from consuming dietary supplements (including anti-oxidants) and anti-inflammatory medications.

The baseline visit (V1) took place after the washout period with subjects in a fasted state (10 hours). During this visit, the subjects underwent a physical examination, had their blood drawn and were randomized into groups.

The laboratory tests and procedures included HS-CRP and a cytokine panel via Sandwich Immunoassay (Panomics, Inc., San Diego, Calif.), Urine F2 isoprostane (Kronos Science Laboratory, Phoenix, Ariz.), and safety laboratory assessments including complete blood count (CBC), comprehensive metabolic panel (CMP), and urinalysis (Primex Labs, Northridge, Calif.) and platelet aggregometry (Plateletworks, Helena Laboratories, Beaumont, Tex.). These lab tests were repeated at the 4 week (V2) and 8 week (V3) visits. An EKG was performed at baseline (V1) and again at 8 weeks (V3). During all 4 visits, anthropomorphic measures (hip and waist measurements, and weight) were obtained and vital signs (blood pressure, temperature, pulse, and respiration) were checked.

Adverse event monitoring was performed at each visit using a standardized set of questions. Subjects were instructed not to change their diet nor start any new exercise program during the course of the study. In order to assess compliance, subjects were required to return all unused investigational product at each visit.

### Outcome Measures

The primary outcome measure of this study was HS-CRP, a marker of cardiac health related to inflammation.

### Randomization

Subjects were randomized using a table of random numbers derived from a random number generating program. A simple randomization schema utilizing 4 groups was given to the clinical staff. A single master sheet was used to randomize subjects if they were eligible for inclusion in the study following the clinical screen. The master sheet included subject number, coordinator initials and date of randomization was used to track the process and insure no duplications of assignment. None of the clinical staff had access to the assignment code key.

### Product Description

The test product was XanGo juice produced by XanGo, LLC. The primary ingredient of XanGo Juice was mangosteen (Garcinia mangostana L.) whole fruit puree. XanGo Juice also contained apple fruit juice, pear fruit juice, grape fruit juice, pear fruit puree, blueberry fruit juice, raspberry fruit juice, strawberry fruit juice, cranberry fruit juice, and cherry fruit juice. The placebo consisted of water, sucrose (3 g/30 ml), citric acid, red grape juice concentrate, fiber complex, grape skin, natural flavors, red #40, cloud (ester gum), whey protein isolate, sodium benzoate, xanthan gum, blue #1, and caramel color.

Three different dosages of the juice were tested and compared to placebo. The product doses tested were 3 oz, 6 oz and 9 oz. All doses and placebo were consumed in a total of 9 oz of liquid in identical bottles. The placebo was used to make up the volume for the lower doses. Subjects were instructed to consume the assigned drink twice a day, once in the morning and again in the evening. They therefore took a total of 0 to 18 oz of active product per day in 18 oz of fluid.

### Data Management

Data was analyzed using paired sample t-tests for within subject means comparisons, independent sample t-tests for between group comparisons (Placebo vs. each of the active groups individually), t-tests of difference scores for both within and between group comparisons (Placebo vs. each of the active groups individually). Data was analyzed with the CRO team continuing the ‘blinded’ structures concerning group assignment. The analysis team only had the Placebo group identified to them so that they would know how to organize the statistical comparisons. Only after the analysis was completed was the ‘blind’ broken in order to properly report relationships between the 3 different strengths of the product being tested and placebo.

Statistical analyses (descriptive statistics and means comparison tests, both within and between group, were performed using SPSS Base System ver. 16 (SPSS Inc., Chicago Ill.)

### Results

One hundred twenty two persons were screened for the study, 44 were randomized and 40 completed the study. Two subjects withdrew from the study due to family related issues, and two subjects were administratively withdrawn for noncompliance. The groups were not significantly different at baseline on age, gender, CRP, BMI, or percentage body fat (Table 2). List wise deletion was utilized in the analysis of study data as each of the 4 groups had a sample size of less than 20.

TABLE 2
Baseline Demographics

| | 6 oz XanGo | 12 oz XanGo | 18 oz XanGo | Placebo |
|---|---|---|---|---|
| N | 11 | 12 | 9 | 8 |
| Male | 1 | 0 | 1 | 0 |
| Female | 10 | 12 | 8 | 8 |
| Age | 52 | 33 | 50 | 45 |
| BMI | 33.7 | 32.6 | 34.1 | 34.8 |
| Body Fat % | 41.5 | 39.3 | 37.8 | 39.3 |

Baseline characteristics of the 40 subjects who completed the study, by study group. Means are given for age, CRP levels, BMI and percentage body fat.

Mean HS-CRP measurements dropped after 8 weeks treatment compared to baseline in all 3 juice product dose groups. In contrast, HS-CRP measurements increased in the placebo group. None of the changes from baseline nor the between group comparisons at each time point were significantly different (Table 3). However the comparison of change from baseline to 8 weeks between the 18 oz group and the placebo group was significant (p=0.019).

TABLE 3
HS-CRP

| Group | Baseline | 8 weeks | Change (8 weeks) |
|---|---|---|---|
| Placebo | 13.10 ± 5.2 | 14.00 ± 8.5 | +0.90 ± 9.5 |
| 6 oz XanGo | 21.30 ± 16.8 | 14.65 ± 8.8 | −6.65 ± 11.7 |
| 12 oz XanGo | 18.33 ± 9.0 | 11.67 ± 4.2 | −6.66 ± 5.8 |
| 18 oz XanGo | 12.00 ± 5.6 | 10.67 ± 5.6 | −1.33 ± 3.0 |

Mean HS-CRP levels plus/minus standard deviations at baseline, after 8 weeks and the change over time. None of the changes from baseline were statistically significant. However the comparison of change from baseline to 8 weeks between the 18 oz group and the placebo group was significant (p = 0.019).

### Discussion

No statistically significant effects on CRP were observed at four weeks. At eight weeks, XanGo juice demonstrated an ability to reduce inflammation (as measured by HS-CRP), at all 3 dosages while the placebo group showed a small increase in the amount of inflammation. When compared with placebo, the change in HS-CRP over 8 weeks was statistically significant in the 18 oz XanGo Juice group, but not for the 6 oz and 12 oz groups. This suggests a possible dose dependent effect, but must be interpreted cautiously as the measurement variability was greater in the lower dose groups.

Other markers of inflammation (inflammatory cytokines) and a marker of lipid peroxidation (a potential indicator of oxidative stress) did not show any clinically significant differences for the juice groups when compared with placebo.

This study was limited by its small sample size. There were 40 subjects in the study and 4 groups, thus there were approximately 10 subjects per group. The small sample size decreases the likelihood that changes from baseline will be significant and reduces the likelihood that between group differences will be statistically significant. In addition, the use of multiple comparisons in the analysis of the study data increases the risk of statistical error resulting in a false positive. The use of an ANOVA or ANCOVA analysis, which would have been more appropriate to compare multiple groups, was precluded by the small sample size and lack of a reliable covariant. Therefore, the results of this pilot study should be interpreted as trends to be confirmed with further investigations using a larger sample size.

The XanGo Juice product was safe at all dosages tested. There were no adverse events (clinical, laboratory, or vital sign) attributed to the product during the course of the study.

### Example 3

### Supplementation with XanGo Juice and Eleviv

The purpose of this study was to evaluate the combined effects of two commercially available dietary supplements versus a look-alike placebo on measures of Mood State, Hormone Balance, and Oxidative/Inflammatory Balance. The dietary supplements are (1) a fruit juice product based on Mangosteen fruit (XanGo Juice), and (2) an herbal supplement containing Citrus sinensis extract, Malaysian Eurycoma longifolia extract, Camellia sinensis extract, and L-theanine (Eleviv).

Both products are currently available over-the-counter and previous studies on each product separately have shown benefits in cellular protection and inflammation (for the Juice product) and hormone balance and mood (for the herbal product). Our hypothesis was that the combination of the two supplements under investigation may generate greater benefits in people who consume them together.

### Procedures

We recruited 30 subjects (22 women and 8 men) to participate in this 4-week supplementation study. Each participant was randomly assigned to receive active supplements (XanGo Juice and Eleviv) or a look-alike placebo of both supplements. Neither the subject nor the investigators knew which supplement was active versus placebo. Both groups consumed 6 ounces daily of the juice (3 oz Am and 3 oz PM) and 2 capsules/day of the herbal product (AM). Mood, hormones and inflammation were monitored using methods well known in the art. Mood outcomes were measured by the Profile of Mood States method, Inflammation was measured using the HS-CRP test, and metabolic balance was measured by the ratio of salivary cortisol to Free Androgen Index.

### Results

There were no significant differences between supplement groups on measures of female reproductive hormones (Progesterone and Estrogen, including Estridiol, Estriol, Estrone, 2-hydroxyestrone, 16-alpha-hydroxysterone, and Estrone sulfate). There were no significant differences between supplement groups on measures of Homocysteine or Lipid Peroxides.

There was a trend in both men and women with elevated Sex Hormone Binding Globulin (SHBG) to experience reductions by the supplementation regimen—and a trend for those with a low Free Androgen Index (FAI) to experience elevations with the supplement regimen.

The results of the primary outcome measures are presented in table 4 below.

TABLE 4
Results

| | Placebo | XanGo + Eleviv |
|---|---|---|
| HS-CRP | | |
| Baseline | 1.64 | 1.86 |
| Post-treatment | 1.35 | 0.77* (−59%) |
| Fibrinogen | | |
| Baseline | 379 | 328 |
| Post-treatment | 367 | 284* (−13%) |
| 24 h Cortisol: Free Androgen Index | | |
| Baseline | 0.227 | 0.417 |
| Post-treatment | 0.256 | 0.099* −61.4%) |
| Vigor | | |
| Baseline | 15.2 | 13.3 |
| Post-treatment | 16.2 | 16.9* (+27%) |
| Tension | | |
| Baseline | 12.0 | 13.9 |
| Post-treatment | 8.5 | 9.2* (−34%) |
| Depression | | |
| Baseline | 11.5 | 13.1 |
| Post-treatment | 7.1* | 7.9* |
| Anger | | |
| Baseline | 9.8 | 11.3 |
| Post-treatment | 6.0* | 7.5* |
| Fatigue | | |
| Baseline | 12.0 | 15.1 |
| Post-treatment | 7.9* | 10.4* |
| Confusion | | |
| Baseline | 8.3 | 8.2 |
| Post-treatment | 6.1* | 6.1* |

*denotes statistical significance (P < 0.05)

Significant differences between supplement groups were found on measures of Inflammation (high-sensitivity C-Reactive Protein and Fibrinogen), metabolic balance (ratio between FAI/Csum=Free Androgen Index/24 h Cortisol exposure), vigor and tension. Changes in the measures of depression, anger, fatigue, and confusion did not reach statistical significance.

Table 5 compares the changes in various outcome measures that occurred after supplementation with XanGo juice alone, Eleviv alone, or the combination of XanGo and Eleviv.

TABLE 5
Comparison of single supplement and combination supplement studies

| | Eleviv Alone | XanGo Alone | Combination |
|---|---|---|---|
| | (8 weeks) | (8 weeks) | (4 weeks) |
| HS-CRP | | −31% | −59% |
| Metabolic Balance # | −15% | | −61.4% |
| Vigor | +29% | | +27% |
| Tension | −19% | | −34% |

The data points represent the change from baseline at the indicated time after each treatment regimen. # Indicates that there are differences between studies in the method of assaying for the outcome measure. The data for XanGo alone represents the change after treatment with six ounces daily of XanGo Juice.

Comparison of the studies of individual dietary supplements with the study of the combination supplement shows that the Eleviv-XanGo Juice combination has an enhanced effect relative to either supplement alone. For example, while XanGo juice shows a decrease in inflammation in 8 weeks (as measured by HS-CRP), the combination shows an even greater effect in only 4 weeks. Furthermore, tension is reduced to a greater degree after treatment with the combination than with Eleviv alone. Also, while different methods were used to collect data regarding metabolic balance in the Eleviv alone and combination studies, Applicants believe that the larger increase in metabolic balance with the combination supplement relative to Eleviv alone is significant (−15% at 8 weeks for Eleviv alone versus −61.4% at 4 weeks in the combination). All of these additional effects are seen in the combination supplement, while the increase in vigor seen with Eleviv alone is also maintained.

It will be obvious to those having skill in the art that many changes may be made to the details of the above-described embodiments without departing from the underlying principles of the invention. The scope of the present invention should, therefore, be determined only by the following claims.

1. A combination supplement comprising Citrus sinensis extract, Eurycoma longifolia extract, Camellia sinensis extract, L-theanine, pericarp from the fruit of a Garcinia mangostana L. tree, and juice from the fruit of a Garcinia mangostana L. tree.

2. The combination supplement of claim 1, wherein the combination supplement comprises a plurality of dietary supplements.

3. The combination supplement of claim 1, wherein the combination supplement comprises a first dietary supplement and a second dietary supplement.

4. The combination supplement of claim 3, wherein the first dietary supplement comprises Citrus sinensis extract, Eurycoma longifolia extract, Camellia sinensis extract, and L-theanine, and wherein the second dietary supplement comprises pericarp from the fruit of a Garcinia mangostana L. tree, and juice from the fruit of a Garcinia mangostana L. tree.

5. The combination supplement of claim 4, wherein the first dietary supplement comprises about 23% to about 30% by weight Citrus sinensis extract.

6. The combination supplement of claim 4, wherein the first dietary supplement comprises about 6% to about 15% by weight Eurycoma longifolia extract.

7. The combination supplement of claim 4, wherein the first dietary supplement comprises about 15% to about 23% by weight Camellia sinensis extract.

8. The combination supplement of claim 4, wherein the first dietary supplement comprises about 6% to about 15% by weight L-theanine.

9. The combination supplement of claim 4, wherein the second dietary supplement comprises about 3% to about 50% by weight pericarp from the fruit of a Garcinia mangostana L. tree.

10. The combination supplement of claim 4, wherein the second dietary supplement further comprises a fruit juice or a vegetable juice.

11. (canceled)

12. A method of supplementing the diet of an individual comprising administering to the individual a combination supplement comprising Citrus sinensis extract, Eurycoma longifolia extract, Camellia sinensis extract, L-theanine, pericarp from the fruit of a Garcinia mangostana L. tree, and juice from the fruit of a Garcinia mangostana L. tree.

13. The method of claim 12, wherein the combination supplement comprises a plurality of dietary supplements.

14. The method of claim 12, wherein the combination supplement comprises a first dietary supplement and a second dietary supplement.

15. The method of claim 14, wherein the first dietary supplement comprises Citrus sinensis extract, Eurycoma longifolia extract, Camellia sinensis extract, and L-theanine, and wherein the second dietary supplement comprises pericarp from the fruit of a Garcinia mangostana L. tree, and juice from the fruit of a Garcinia mangostana L. tree.

16. The method of claim 15, wherein the first dietary supplement is administered once a day, and wherein the second dietary supplement is administered twice a day.

17. The method of claim 15, wherein the first dietary supplement and the second dietary supplement are administered before noon on the day of administration, and the second dietary supplement is administered after noon on the day of administration.

18. The method of claim 17, wherein administering the combination supplement to the individual results in at least one of the following changes in the individual: increased vigor, decreased tension, improved metabolic balance, or decreased C-reactive protein.

19. (canceled)

20. A kit comprising a combination supplement and instructions for use of the combination supplement, wherein the combination supplement comprises Citrus sinensis extract, Eurycoma longifolia extract, Camellia sinensis extract, L-theanine, pericarp from the fruit of a Garcinia mangostana L. tree, and juice from the fruit of a Garcinia mangostana L. tree.

21. The kit of claim 20, wherein the kit comprises a first dietary supplement and a second dietary supplement.

22. The kit of claim 21, wherein the first dietary supplement comprises Citrus sinensis extract, Eurycoma longifolia extract, Camellia sinensis extract, and L-theanine, and wherein the second dietary supplement comprises pericarp from the fruit of a Garcinia mangostana L. tree, and juice from the fruit of a Garcinia mangostana L. tree.

23. (canceled)
